(19) United States
(12) Patent Application Publication
(10) Pub. No.: US 2023/0340140 A1
(43) Pub. Date: Oct. 26, 2023

(54) SUBCUTANEOUS ANTI-C5AR ANTAGONIST TREATMENT REGIMEN WITH AVDORALIMAB

(71) Applicant: INNATE PHARMA, MARSEILLE (FR)

(72) Inventor: CARINE PATUREL, MARCY L'ETOILE (FR)

(21) Appl. No.: 18/020,954

(22) PCT Filed: Aug. 6, 2021
(86) PCT No.: PCT/EP2021/072027

§ 371 Date: Feb. 13, 2023

Related U.S. Application Data
(60) Provisional application No. 63/064,442, filed on Aug. 12, 2020.

(51) Int. Cl. C07K 16/28; A61P 37/00

(52) U.S. Cl. CPC C07K 16/2896; A61P 37/00; A61K 2039/505

(57) ABSTRACT

The present invention concerns therapeutic uses of C5aR antagonists, particularly avdoralimab, in particular in relation to chronic inflammatory or autoimmune disorders, and in particular in inflammatory skin diseases.

### CROSS-REFERENCE TO RELATED APPLICATIONS

This application claims the benefit of U.S. Provisional Application No. 63/064,442 filed 12 Aug. 2020; which is incorporated herein by reference in its entirety; including any drawings.

### REFERENCE TO SEQUENCE LISTING

The present application is being filed along with a Sequence Listing in electronic format. The Sequence Listing is provided as a file entitled “C5aR SC_ST25”, created 5 Aug. 2021, which is 22 KB in size. The information in the electronic format of the Sequence Listing is incorporated herein by reference in its entirety.

### TECHNICAL FIELD

The present invention concerns therapeutic uses of C5aR antagonists in particular in relation to chronic inflammatory or autoimmune disorders, and in particular in inflammatory skin diseases.

### BACKGROUND

Proteolysis of each of the complement proteins C3-C5 gives rise to amino-terminal cationic fragments with signalling molecules called anaphylatoxins. The most potent of these, C5a, elicits the broadest responses. Considering the components of the inflammatory response as margination and infiltration of leukocytes, release of granule-bound proteolytic enzymes, production of activated oxygen and nitrogen-derived radicals, changes in blood flow and capillary leakage, along with the ability to contract smooth muscle, the C5a molecule is the “complete” pro-inflammatory mediator. At sub-nanomolar to nanomolar levels, the C5a molecule elicits chemotaxis of all myeloid lineages (neutrophils, eosinophils and basophils, macrophages and monocytes), and causes vascular permeability which is markedly potentiated by prostaglandins and circulating leukocytes. Higher nanomolar concentrations elicit degranulation and activation of NADPH oxidase. This breadth of bioactivity contrasts with other inflammatory mediators.

The activities of C5a are mediated by the binding of the C5a to its receptor C5aR, also referred to as C5aR1. C5aR belongs to the family of seven transmembrane G-protein-coupled receptors. C5aR is a high affinity receptor for C5a, with a Kd of ˜1 nM, and is located on a number of different cell types including leukocytes. The number of receptors per cell is extremely high, up to 200,000 sites per leukocyte. Biological activation of the receptor occurs over the range that saturates binding. In skin, C5aR is expressed in particular on mast cells and neutrophils, and C5a can mediate the activation and/or migration of the cells. Neutrophils are a type of phagocyte normally found in the bloodstream, and during the beginning (acute) phase of inflammation, neutrophils are one of the first responders of inflammatory cells to migrate toward the site of inflammation. Mast cells are long-living tissue-resident immune cells that migrate to and differentiate within the tissue.

The C5aR structure conforms to the seven transmembrane receptor family, with the extracellular N-terminus being followed by seven transmembrane helices connected by interhelical domains alternating as intracellular and extracellular loops, and ending with an intracellular C-terminal domain. C5aR contains an extended N-terminal extracellular domain. This large N-terminal domain is typical of G-protein coupled receptors which bind peptides including the IL-8 and fMet-Leu-Phe (FMLP) receptor families.

Inhibition of the C5a responses with C5aR antagonists reduces the acute inflammatory response mediated via C5a without affecting other complement components. To this end, anti-C5a receptor rantagonist antibodies have been previously described. For example, WO 95/00164 describes antibodies directed against an N-terminal peptide (residues 9-29) of C5aR. WO 03/062278 also describes antibodies directed against C5aR. Antibodies directed against C5aR were shown to have excellent properties, such as being very effective at blocking C5a binding to its receptor, stopping C5a-directed migration of neutrophils in vitro, and preventing inflammation in animal models. More recently, WO2012/168199 describes a series of human antibodies including antibody 32F3A6 GL directed against C5aR that can be used to control chronic diseases by administering the antibody on successive occasions over months or years with decreased risk of eliciting antibodies directed against the mouse antibodies (the HAMA response).

C5a is involved in the pathogenesis of various disorders including rheumatoid arthritis, psoriasis, sepsis, reperfusion injury, and adult respiratory distress syndrome (Gerard and Gerard, 1994 Annu Rev Immunol (12): 775-808; Murdoch and Finn (2000) Blood 95 (10): 3032-3043).

More recently, C5a has also been found to be associated with cutaneous disorders, including in particular cutaneous disorders in which mastocytes play a major role in driving disease.

To date, however, no treatment regimens have been developed for anti-C5aR antibodies that are suitable for such disease. There is therefore a need for treatment regimens suitable for anti-C5aR antibodies.

### SUMMARY

This disclosure provides regimens and methods of using blocking antibodies that bind C5aR (C5aR1) in the treatment of disease, in particular chronic inflammatory disease, and including in the treatment of inflammatory skin disease indications where such C5aR antagonists have utility. The anti-C5aR antibodies can be particularly advantageously used for example to treat inflammatory skin disease in which mast cells are contributing to inflammation, particularly where they are activated or degranulating. The anti-C5aR1 antibodies can also be used for example to treat inflammatory skin disease in which neutrophils are activated and/or are generally contributing to inflammation. The treatments can be used advantageously to treat chronic inflammatory skin disease where efficacy must be accompanied by minimal side effects (high safety), and yet further in inflammatory skin diseases characterized by acute phases or exacerbations where blockade of the inflammation must be achieved in a short time frame. The regimens disclosed herein can be administered subcutaneously and in a series of flat doses for all individuals (e.g. irrespective of body weight or surface area).

One aspect of the disclosure relates to the use of the C5aR (C5aR1) antagonist antibodies, e.g. avdoralimab, for treatment or prevention of an inflammatory disease (e.g., a skin disease, rheumatoid arthritis), wherein the C5aR antagonist antibody is administered in a plurality of flat doses that permits the maintenance of a targeted blood concentration around a close range for the duration of the treatment, and that can reach the targeted blood concentration within the first day from treatment. The targeted blood concentration is designed to provide substantially full blockade of C5aR1 on mast cells and/or neutrophils, including in skin. The flat dose treatment regimen will be useful across a population of individuals (e.g. adults) to obtain and maintain a blood concentration that provides substantially full blockade of C5aR on mast cells and/or neutrophils, particularly mast cells present in the skin. The treatment furthermore does not result in depletion or phagocytosis of mast cells and/or neutrophils. In one embodiment, the C5aR antagonist antibody is administered subcutaneously in a dose of 400-600 mg, optionally 300-600 mg, optionally 400-500 mg, optionally 450-500 mg, optionally 425-475 mg, optionally about 450 mg. In one embodiment, the C5aR antagonist antibody is administered about once per week.

One aspect of the disclosure provides a method of administering an antagonist anti-C5aR1 antibody to an individual in need thereof (e.g. an individual having an inflammatory skin disease), comprising administering to the individual the antagonist anti-C5aR1 antibody (e.g. avdoralimab or a function conservative variant thereof), wherein the doses are administered subcutaneously in an amount of 400-500 mg, optionally 450 mg, once per week. Optionally, it can be specified that the method comprises administering a plurality of doses of the antagonist anti-C5aR1 antibody.

Optionally, it can be specified that all administrations of the C5aR (C5aR1) antagonist comprise the same amount of C5aR antagonist. For example, each dose, including the initial dose and all subsequent doses are administered in an amount of 450 mg.

The effect of treatment using a C5aR antagonist such as an anti-C5aR1 antibody (e.g. avdoralimab or a functionally conservative variant thereof) can be useful for providing a fast response, for example to observe a therapeutic effect in about one month, optionally two week, or less. A treatment of the disclosure (e.g. that provides a fast response) can be particularly advantageous to treat or prevent acute phases and/or exacerbations in an inflammatory disease, for example an inflammatory skin disease, for example psoriasis, acne vulgaris, hidradenitis suppurativa (HS), systemic or cutaneous lupus erythematosus, cutaneous small vessel vasculitis, urticaria or urticarial vasculitis or bullous pemphigoid. In one embodiment the present invention relates to a C5aR antagonist for use in a method of reducing or preventing acute inflammation, wherein an effect is obtainable after 4, 3, 2 or 1 dosage(s). In one embodiment the present invention relates to a C5aR antagonist for use in a method of reducing or preventing skin inflammation characterized by acute phase(s) and/or an exacerbation(s), wherein an effect is obtainable within a month after the first administration of the C5aR antagonist.

The effect of treatment using a C5aR antagonist (e.g. avdoralimab) can be useful for providing a response in tissues (e.g. skin), for example to obtain a reduction of inflammation in tissues (e.g. skin) for a long duration of time, e.g. for at least 3, 6, 9 or 12 months. In one embodiment the present invention relates to a C5aR antagonist (e.g. an anti-C5aR1 antibody, avdoralimab) for use in a method of reducing or preventing skin inflammation, wherein an effect is obtainable after 4, 3, 2 or 1 dosage(s). In one embodiment the present invention relates to a C5aR antagonist for use in a method of reducing or preventing skin inflammation, wherein an effect is obtainable within a week (e.g. 7, 6, 5, 4, 3, 2 or 1 days) after the first administration of avdoralimab.

In one embodiment, the treatment of the disclosure achieves, within a 24, 12 or 6 hour period from first administration, a targeted blood concentration of antibody of at least about 20 μg/ml. In one embodiment, the treatment of the disclosure achieves, starting from the fourth or fifth administration, a blood concentration of antibody of at least 20 μg/ml, optionally at least 50 μg/ml, optionally at least 70 μg/ml, is maintained for the duration of the treatment. In one embodiment, the treatment of the disclosure provides that the blood concentration of antibody not exceed the concentration that provides EC₁₀₀ for inhibition of C5aR activity on neutrophils in tissues (e.g. 100 μg/ml) by more than 2-fold or optionally by more than 50%, 30% or 20% during the treatment (e.g. for the duration of any 24 or 48 hour period).

In one embodiment, the functionality of avdoralimab may be considered linked to the ability of avdoralimab to antagonize C5aR (C5aR1) either by inhibiting or reducing binding of C5a to C5aR (C5aR1) and/or by inhibiting or reducing C5aR (C5aR1) mediated biological effect of C5a, such as a) C5a induced mast cell and/or neutrophil activation, b) C5a induced cell migration and/or c) C5a induced neutrophil maturation.

Avdoralimab can be specified as being administered as part of a pharmaceutical composition optionally including one or more pharmaceutical excipient. In one aspect the invention relates to a method for treatment or prevention of disease can be specified as comprising administering a therapeutically effective amount of avdoralimab as described herein, to an individual in need.

In one embodiment, provided is a method for treating an individual, the method comprising administering to the individual, by subcutaneous administration, a dose of 450 mg of avdoralimab once per week. In one embodiment, provided is a method inhibiting the activity of mast cells and/or neutrophils in skin tissue in an individual, the method comprising administering to the individual avdoralimab by subcutaneous administration, for example a dose of 450 mg of avdoralimab once per week. In one embodiment, provided is a method treating or preventing acute skin inflammation or an exacerbation of skin inflammation in an individual, the method comprising administering to the individual avdoralimab by subcutaneous administration, for example a dose of 450 mg of avdoralimab once per week.

Further aspects are described herein in the section titled “Embodiments”.

### BRIEF DESCRIPTION OF DRAWINGS

FIG. 1 shows the mean avdoralimab (IPH5401) serum concentration (μg/mL) over time after single s.c. dosing of subjects with rheumatoid arthritis (RA).

FIG. 2 shows the mean avdoralimab serum concentration (μg/mL) over time after multiple s.c. dosing of subjects with RA.

FIG. 3 shows the mean C5aR occupancy (%) on neutrophils over time by s.c. dose levels (single s.c. administration).

FIG. 4 shows the mean C5aR occupancy (%) on neutrophils and granulocytes over time by s.c. dose levels (multiple s.c. administrations).

FIG. 5 shows the model developed to describe the observed PK of avdoralimab following repeated intravenous administration in humans.

FIG. 6 shows C5a-induced CD11 b expression on neutrophils (defined as SSChigh CD16+ cells) was quantified as median fluorescence intensity (MedFI) of PE-Cy5 for a given Avdoralimab concentration.

FIG. 7 shows results of PK prediction for Q1w avdoralimab for the 75 mg, 150 mg, 300 mg, 450 mg, 600 mg flat doses.

### DESCRIPTION

As used in the specification, “a” or “an” may mean one or more. As used in the claim(s), when used in conjunction with the word “comprising”, the words “a” or “an” may mean one or more than one. As used herein “another” may mean at least a second or more.

Where “comprising” is used, this can optionally be replaced by “consisting essentially of” or by “consisting of”.

Whenever within this whole specification “treatment or prevention of a disorder” or the like is mentioned with reference to C5aR antagonist (e.g., anti-C5aR1 antibody, avdoralimab), there is meant: (a) method of treatment of the disorder, said method comprising the step of administering (for at least one treatment) a C5aR antagonist, (preferably in a pharmaceutically acceptable carrier material) to an individual, a mammal, especially a human, in need of such treatment, in a dose that allows for the treatment of the disorder, (a therapeutically effective amount), preferably in a dose (amount) as specified herein; (b) the use of an C5aR antagonist for the treatment or prevention of the disorder, or an C5aR antagonist, for use in said treatment (especially in a human); (c) the use of an C5aR antagonist for the manufacture of a pharmaceutical preparation for the treatment of the disorder, a method of using an C5aR antagonist for the manufacture of a pharmaceutical preparation for the treatment of the disorder, comprising admixing an C5aR antagonist with a pharmaceutically acceptable carrier, or a pharmaceutical preparation comprising an effective dose of an C5aR antagonist that is appropriate for the treatment of the disorder; or (d) any combination of a), b), and c), in accordance with the subject matter allowable for patenting in a country where this application is filed.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The term “specifically binds to” means that an antibody can bind preferably in a competitive binding assay to the binding partner, e.g., C5aR (C5aR1), as assessed using either recombinant forms of the proteins, epitopes therein, or native proteins present on the surface of isolated target cells. Competitive binding assays and other methods for determining specific binding are further described below and are well known in the art.

When an antibody is said to “compete with” a particular monoclonal antibody, it means that the antibody competes with the monoclonal antibody in a binding assay using either recombinant C5aR (C5aR1) molecules or surface expressed C5aR (C5aR1) molecules. For example, if a test antibody reduces the binding of a reference antibody to a C5aR polypeptide or C5aR-expressing cell in a binding assay, the antibody is said to “compete” respectively with the reference antibody.

The term “hypervariable region” when used herein refers to the amino acid residues of an antibody that are responsible for antigen binding. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917), or a similar system for determining essential amino acids responsible for antigen binding. Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, MD).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein (e.g. heavy or light chains, or CDRs or variable regions thereof) to which it is compared.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

### Methods of Treatment

Compounds or drugs that inhibit or reduce a biological response usually elicited by ligand-receptor interaction are termed receptor antagonists. Such receptor antagonist will bind the receptor but the interaction will not have efficacy. Presence of an antagonist will thus inhibit or reduce the biological effect of the ligand (or ligands) of the receptor. The action of antagonists may be medicated by binding an active site of the receptor thereby blocking or disrupting ligand interaction. Alternatively an antagonist may bind the receptor at a different site which also effective prevents ligand binding or receptor signaling.

The amino acid sequence of the human C5aR (C5aR1) protein is shown below, with the 2^{nd} lop (2^{nd} extracellular loop) indicated by underlining of residues 171-206.

Human C5aR (C5aR1):
(SEQ ID NO: 1)
MDSFNYTTPD YGHYDDKDTL DLNTPVDKTS NTLRVPDILA LVIFAVVFLV GVLGNALVVW
VTAFEAKRTI NAIWFLNLAV ADFLSCLALP ILFTSIVQHH HWPFGGAACS ILPSLILLNM
YASILLLATI SADRFLLVFK PIWCQNFRGA GLAWIACAVA WGLALLLTIP SFLYRVVREE
YFPPKVLCGV DYSHDKRRER AVAIVRLVLG FLWPLLTLTI CYTFILLRTW SRRATRSTKT
LKVVVAVVAS FFIFWLPYQV TGIMMSFLEP SSPTFLLLKK LDSLCVSFAY INCCINPIIY
VVAGQGFQGR LRKSLPSLLR NVLTEESVVR ESKSFTRSTV DTMAQKTQAV.

C5aR antagonists, and in particular antibodies that bind to the second extracellular loop of the human C5aR protein and inhibit the binding of C5a to C5aR have been found to be of relevance in treatment of inflammatory diseases and disorders based on results from several animal models of human disease. The signaling mediated by the C5a receptor upon binding of C5a may thus be inhibited or reduced, resulting an inhibition or reduction of the on-going inflammation process and thus relief of associated conditions.

In any aspect herein, the treatments described herein using a C5aR antagonist can be specified as being for use as a medicament, or for treatment or prevention of disease, in an individual who has an inflammatory disease, for example rheumatoid arthritis or an inflammatory skin disease, for example a chronic inflammatory skin disease in which mast cells (mastocytes) are contributing to inflammation, particularly where they are activated or degranulating, and/or in which neutrophils are activated and/or are generally contributing to inflammation. Optionally, the inflammatory disease is in an acute phases and/or is characterized by exacerbations.

In one embodiment of any aspect herein, the inflammatory skin disease is psoriasis.

In one embodiment of any aspect herein, the inflammatory skin disease is acne vulgaris.

In one embodiment of any aspect herein, the inflammatory skin disease is hidradenitis suppurativa (HS).

In one embodiment of any aspect herein, the inflammatory skin disease is systemic or cutaneous lupus erythematosus.

In one embodiment of any aspect herein, the inflammatory skin disease is cutaneous small vessel vasculitis.

In one embodiment of any aspect herein, the inflammatory skin disease is urticaria or urticarial vasculitis. In one embodiment of any aspect herein, the urticaria is chronic (e.g., chronic spontaneous urticaria; chronic inducible urticaria; urticaria characterized by recurrent urticarial lesions persisting for a period beyond 6 weeks).

In one embodiment of any aspect herein, the inflammatory skin disease is bullous pemphigoid.

In one embodiment of any aspect herein, the inflammatory skin disease is mucous membrane pemphigoid, epidermolysis bullosa acquisita, dermatitis herpetiformis or bullous systemic lupus erythematosus.

In a further embodiment a C5aR antagonist is for use in a method for treatment or decreasing, inhibiting, preventing, halting and/or reversing inflammation (e.g. skin inflammation) in an individual having an inflammatory disease (e.g. an inflammatory skin disease), or to decrease, inhibit, prevent, halt and/or reverse exacerbations and/or acute phases, or skin manifestations, of such disease, to obtain a fast and/or long duration of response (e.g. within one month, two weeks, within one week, or less; lasting for month than one month, three months, six months). In one embodiment the C5aR antagonist is for use in a method for treatment or prevention of psoriasis, acne vulgaris, hidradenitis suppurativa (HS), systemic or cutaneous lupus erythematosus, cutaneous small vessel vasculitis, urticaria or urticarial vasculitis or bullous pemphigoid, for obtaining a fast response. In such an embodiment, a fast response is considered present if it is detectable after a limited number of dosages, such as after at most 5 dosages, such as at most 3 dosages, or preferably after as few as two or even one dosage of the C5aR antagonist. In one embodiment, the methods comprise assessing or detecting a response before the 6th dosage is to be applied, such as after the 5^{th}, 4^{th}, 3^{rd} or 2^{nd} dosage is administered. In one embodiment the response may be detectable before a second dosage is to be administered. In one embodiment, a method of the disclosure can optionally comprise a step of assessing response to treatment (e.g., assessing or detecting an amelioration in inflammatory disease) within 2 months, 1 month or two weeks of first administration of the C5aR antagonist, and upon a determination of an amelioration in inflammatory disease, continuing administration of the C5aR antagonist (e.g. according to the same administration regimen). In one embodiment, a method of the disclosure can optionally comprise a step of assessing an amelioration in inflammatory skin disease within 2 months, 1 month or two weeks of first administration of the C5aR antagonist, and upon a determination of an amelioration in inflammatory skin disease, continuing administration of the C5aR antagonist (e.g. according to the same administration regimen).

The response and/or duration thereof may be detected by various means known to the skilled artisan, as considered relevant for the disease and effect sought.

The response may for example be detected by observing physical symptoms of skin inflammation, or be detected by measurement of relevant disease biomarkers, such as markers describing the disease activity, such as cytokines and chemo-attractants. Markers addressing the presence of specific cell types, such mastocyte, MPO detecting neutrophils, as well as markers of tissue remodelling and/or extracellular matrix homeostasis may be used to evaluate if an early response is obtained. For clarity is noted that the fast response may be considered obtained also in situations where the effect is not actually measured e.g. situations where a response would have been detected if the relevant measurement had been performed.

In one embodiment a fast response is detected (or detectable) by measuring relevant markers locally in the skin or in the periphery, where peripheral signals are preferably detectable in a serum sample.

In one embodiment the C5aR antagonist is for use in a method of treatment of disease seeking a fast response as describe above wherein a relevant change of a mast cell marker, a neutrophil marker, a cytokine marker, a chemoattractant markers or a tissue remodelling marker can be observed.

In one embodiment the C5aR antagonist is for subcutaneous administration.

In one embodiment the C5aR antagonist is for weekly administration, e.g., a treatment comprises administration of avdoralimab every week. In a further embodiment the course of treatment with the C5aR antagonist is for about or at least 2 weeks, for about or at least about 1 month, for about or at least about 2 months, for about or at least about 3 months, for about or at least about 4 months, for about or at least about 5 months or for about or at least about 6 months.

In one embodiment, the C5aR antagonist is administered according to a schedule and in an amount (dose) that maintains, for the duration of the course of treatment (e.g. at least two weeks, three weeks, one month, three months), a concentration in circulation (e.g. the targeted blood concentration), optionally in an extravascular tissue of interest (e.g., in skin), that is higher than the EC₅₀, optionally the EC₇₀, optionally the EC₉₀ or optionally the EC₁₀₀, for inhibition of C5aR (C5aR1) activity in vitro, optionally in C5aR-expressing cells (e.g. mast cells and/or neutrophils), for example as determined by assessing calcium flux, CD11b upregulation, CD62L down regulation and/or cellular (e.g. neutrophil) migration. Optionally the concentration maintained is at least 50% higher than the EC₇₀, optionally EC₉₀ or optionally EC₁₀₀, for binding to C5aR-expressing cells. Optionally the concentration maintained is at least 2, 3, 4, 5 or 6-fold the EC₅₀, for inhibition of C5aR activity in vitro. “EC₅₀” with respect to a particular activity, refers to the efficient concentration of antibody which produces 50% (or 70% or 90% for EC₇₀ and EC₉₀ respectively) of its maximum response or effect with respect to the particular activity. Optionally the concentration in circulation (targeted blood concentration) maintained (e.g. upon completion of the first five administrations) is about or is at least about 60, 70, 80, 90 or 100 μg/ml.

In one embodiment, the C5aR antagonist is administered according to a schedule and in an amount (dose) that maintains, from the third administration forward, a trough concentration (C_{trough}; the lowest concentration reached by the drug before the next dose is administered) of at least 50 μg/mL at steady state in in circulation (e.g. in serum). In one embodiment, following the fifth administration (from the 5th administration onwards), the treatment maintains a trough concentration of at least 70 μg/mL at steady state in circulation (e.g. in serum). In one embodiment, following the fifth, optionally sixth, administration (from the 5th, optionally 6th, administration onwards), the treatment provides a concentration, optionally a trough concentration, of at least 80 μg/mL in skin tissue. In one embodiment, the C5aR antagonist is administered according to a schedule and in an amount (dose) that maintains said trough concentration between each dose of C5aR antagonist administered (e.g. the concentration is maintained for at least one week). In one embodiment, the C5aR antagonist is administered according to a schedule and in an amount (dose) that maintains said trough concentration for the duration of the course of treatment (e.g. at least one month, at least three months).

In one embodiment, the C5aR antagonist is administered according to a schedule and in an amount (dose) wherein the antibody blood concentration does not exceed the targeted blood concentration (e.g. 100 μg/ml or about 100 μg/ml) by more than 2-fold or more than 50% during the treatment, e.g. during any 24 or 48 hour period. The ability of regimen to achieve and/or maintain a particular blood concentration can be assessed (e.g., by observation, by modelling) in a plurality or population of adult individuals, e.g., a population of individuals having body weight and/or surface representative of the human adult population.

One aspect of the disclosure relates to a method for treatment or prevention of disease in an individual comprising administering a therapeutically effective amount of a C5aR antagonist (e.g. anti-C5aR1 antibody, avdoralimab, an antibody having the heavy and light chains CDRs of avdoralimab, a function-conservative variant of avdoralimab) to an individual in need (e.g., an individual having an inflammatory skin disease), wherein the C5aR antagonist is administered as a course of treatment having a long duration, e.g., 1 to 6 months or more.

The embodiments described herein are useful for administration to individuals who are at significant risk or susceptibility to a mast cell and/or neutrophil mediated skin inflammation (e.g., psoriasis, acne vulgaris, hidradenitis suppurativa (HS), systemic or cutaneous lupus erythematosus, cutaneous small vessel vasculitis, urticaria or urticarial vasculitis or bullous pemphigoid, or an acute phase or exacerbation of any of the foregoing).

The embodiments described herein are particularly useful for treatment or prevention of a disease or disorder where delaying, halting and/or inhibition of inflammation or progression of skin inflammation (e.g. severe skin inflammation, psoriasis, acne vulgaris, hidradenitis suppurativa (HS), systemic or cutaneous lupus erythematosus, cutaneous small vessel vasculitis, urticaria or urticarial vasculitis or bullous pemphigoid) is beneficial to the individual. In one embodiment the method for treatment or prevention of skin inflammation, a therapeutically effective amount of a C5aR antagonist to an individual in need, wherein said C5aR antagonist is administered subcutaneously in a course of treatment that has a duration of at least one month, at least two months, at least three months. In one embodiment, each administration of the first administration of C5aR antagonist is a loading dose, and one or a plurality of C5aR antagonist, including the first (initial) dose and the subsequent doses comprise the same amount of C5aR antagonist.

In one embodiment, the dose is a flat dose suitable across a plurality of population of individuals, e.g. irrespective of body weight or body surface area, for example the dose can be a dose of 400-600 mg, optionally 400-500 mg, optionally about 450 mg). In another embodiment, the dose is 6 mg/kg body weight.

In any embodiment herein, the dose is administered weekly. It can be specified that the doses can be repeated once every week for a plurality of administrations. Optionally the course of the treatment is at least one month, two months or three months.

In one embodiment the invention relates to a method for treatment or prevention of inflammation comprising administering a therapeutically effective amount of avdoralimab to an individual in need, wherein a fast response is detectable. As described herein above a fast response may refer to a situation where a relevant change of a mast cell marker (e.g. mast cell activation marker), a neutrophil marker (e.g. neutrophil activation marker), as cytokine marker, a chemoattractant markers or a tissue remodelling marker can be observed after a limited number of dosage or a limited time from 1^{st} administration.

### C5aR Antagonists

C5a, the A fragment of complement factor 5, binds its receptor C5aR (also referred to as C5aR1) and stimulates the inflammatory response. Inhibition of the C5a response with C5aR antagonists reduces the acute inflammatory response mediated via C5a without affecting other complement components. Different types of C5aR antagonists have previously been described (see background section) including peptide molecules such as cyclic peptide and anti-C5a receptor antibodies.

Avdoralimab is an antibody that binds the 2^{nd} loop of C5aR (also referred to as the 2^{nd} extracellular loop) and that acts as a C5aR antagonist. In one embodiment the C5aR antagonist is an antibody binding the 2^{nd} loop of human C5aR and interacts (binds) specifically with C5aR, and not with other receptors such as C5L2, which is also a receptor for C5a. A C5aR (C5aR1) antagonist specifically binds C5aR (C5aR1). In particular, the C5aR antagonist does not bind to C5L2 (C5aR2). This interaction leading to inhibition or reduction of C5a binding to C5aR.

The heavy and light chains of avdoralimab (see WHO Drug Information, Vol. 33, No. 2, 2019) are shown in SEQ ID NOS: 2 and 3, and below.

| Avdoralimab Heavy chain | |
|---|---|
| (SEQ ID NO: 2) | |
| EVQLVESGGG LVQPGGSLRL SCAASGFTFS SYVMHWVRQA TGKGLEWVSA | 50 |
| IDTGGGTYYA DSVKGRFTIS RENAKNSLYL QMNSLRAGDT AVYYCARDYY | 100 |
| YYASGSYYKA FDIWGQGTMV TVSSASTKGP SVFPLAPSSK STSGGTAALG | 150 |
| CLVKDYFPEP VTVSWNSGAL TSGVHTFPAV LQSSGLYSLS SVVTVPSSSL | 200 |
| GTQTYICNVN HKPSNTKVDK RVEPKSCDKT HTCPPCPAPE AEGAPSVFLF | 250 |
| PPKPKDTLMI SRTPEVTCVV VDVSHEDPEV KFNWYVDGVE VHNAKTKPRE | 300 |
| EQYNSTYRVV SVLTVLHQDW LNGKEYKCKV SNKALPSSIE KTISKAKGQP | 350 |
| REPQVYTLPP SREEMTKNQV SLTCLVKGFY PSDIAVEWES NGQPENNYKT | 400 |
| TPPVLDSDGS FFLYSKLTVD KSRWQQGNVF SCSVMHEALH NHYTQKSLSL | 450 |
| SPGK. | |
| Avdoralimab light chain | |
| (SEQ ID NO: 3) | |
| EIVLTQSPGT LSLSPGERAT LSCRASQSVS SRYLAWYQQK PGQAPRLLIY | 50 |
| GASSRATGIP DRFSGSGSGT DFTLTISRLE PEDFAVYYCQ QYGSPLTFGQ | 100 |
| GTKLEIKRTV AAPSVFIFPP SDEQLKSGTA SVVCLLNNFY PREAKVQWKV | 150 |
| DNALQSGNSQ ESVTEQDSKD STYSLSSTLT LSKADYEKHK VYACEVTHQG | 200 |
| LSSPVTKSFN RGEC. | |

Avdoralimab, or more generally an antibody having the heavy chain variable region of SEQ ID NO: 4 and a light chain variable region of SEQ ID NO: 8, binds the 2^{nd} loop of C5aR (also referred to as the 2^{nd} extracellular loop) and that acts as a C5aR antagonist. Avdoralimab does not bind to the C5aR2 protein. Avdoralimab comprises the VH and VL domains, and CDRs, of antibody 32F3A6. In some embodiments, the C5aR antagonist (e.g., antibody) comprises H-CDR1, H-CDR2 and/or H-CDR3 sequences derived from the V_{H} having the amino acid sequence of SEQ ID NO: 4. In some embodiments, the C5aR antagonist (e.g., antibody) comprises L-CDR1, L-CDR2 and/or L-CDR3 sequences derived from the VL having the amino acid sequence of SEQ ID NO: 8. In some embodiments, the C5aR antagonist (e.g., antibody) comprises H-CDR1, H-CDR2 and/or H-CDR3 sequences, as determined according to Kabat numbering, of the VH having the amino acid sequence of SEQ ID NO: 4, and L-CDR1, L-CDR2 and/or L-CDR3 sequences, as determined according to Kabat numbering, of the VL having the amino acid sequence of SEQ ID NO: 8. In one embodiment, the C5aR antagonist (e.g., antibody) comprises H-CDR1, H-CDR2 and/or H-CDR3 sequences of SEQ ID NOS: 5, 6 and 7 respectively, and L-CDR1, L-CDR2 and/or L-CDR3 sequences of SEQ ID NOS: 9, 10 and 11 respectively.

In some embodiments, the C5aR antagonist is an antibody comprising a H-CDR1 corresponding to Kabat residues 31-35 of SEQ ID NO: 4, a H-CDR2 corresponding to Kabat residues 50-65 of SEQ ID NO: 4, and a H-CDR3 corresponding to Kabat residues 95-102 according to Kabat) of SEQ ID NO: 4. In some embodiments, an anti-C5aR antibody is an antibody comprising a L-CDR1 corresponding to Kabat residues 24-34 of SEQ ID NO: 8, a L-CDR2 corresponding to Kabat residues 50-56 of SEQ ID NO: 8, and an L-CDR3 corresponding to Kabat residues 89-97 of SEQ ID NO: 8. Optionally, a CDR may comprise one, two, three, four, or more amino acid substitutions.

In some embodiments, the C5aR antagonist is or comprises an antibody wherein the variable region of the heavy chain of said antibody comprises a sequence at least 96, 97, 98 or 99% identical to SEQ ID NO: 4 and/or wherein the variable region of the light chain of said antibody comprises a sequence at least 96, 97, 98 or 99% identical to SEQ ID NO: 8.

32F3A6 VH
(SEQ ID NO: 4)
EVQLVQSGGGLVHPGGSLRLSCAGSGFTFSSYVMHWVRQAPGKGLEWVS
AIDTGGGTYYADSVKGRFTISRDNAKNSLYLQMNSLRAEDMAVYYCARD
YYYYASGSYYKAFDIWGQGTMVTVSS.
32F3A6 heavy chain CDRs (according to Kabat
numbering):
HCDR1:
(SEQ ID NO: 5)
SYVMH.
HCDR2:
(SEQ ID NO: 6)
AIDTGGGTYYADSVK.
HCDR3:
(SEQ ID NO: 7)
DYYYYASGSYYKAFDI.
32F3A6 VL
(SEQ ID NO: 8)
EIVLTQSPGTLSLSPGERATLSCRASQSVSSRYLAWYQQKPGQAPRLLI
YGASSRATGIPDRFSGSGSGTDFTLTISRLEPEDFAVYYCQQYGSPLTF
GQGTKLEIK.
32F3A6 light chain CDRs (according to Kabat
numbering):
LCDR1:
(SEQ ID NO: 9)
RASQSVSSRYLA.
LCDR2:
(SEQ ID NO: 10)
GASSRAT.
LCDR3:
(SEQ ID NO: 11)
QQYGSPLT.

The functionality of an anti-C5aR antagonist antibody is dependent on the ability of said antagonist to significantly inhibit or reduce binding of C5a to C5aR, in particular by binding of the anti-C5aR antagonist to C5aR. This may be determined by a displacement assay (SPA), for example as described in Example 2 of WO2012/168199 the disclosure of which is incorporated herein by reference, from which EC₅₀ values can be determined. In one embodiment, the EC₅₀ is below 50 nM. In a further embodiment of the invention the C5aR antagonist displaces C5a in an SPA assay, with an EC₅₀ below 50 nM, such as below nM, such as below 30 nM, such as below 20 nM, such as below 10 nM, such as below 5 nM or even below 4 nM, or with and EC₅₀ below 3 nM or even below 2.5 nM or 2.0 nM.

The term binding “affinity” is used to describe monovalent interactions (intrinsic activity). Binding affinity between two molecules, e.g. an antagonist and a receptor, through a monovalent interaction may be quantified by determination of the dissociation constant (K_{D}) by measurement of the kinetics of complex formation and dissociation, e.g. by surface plasmon resonance (SPR) method. The rate constants corresponding to the association and the dissociation of a monovalent complex are referred to as the association rate constant kₐ (or kₒₙ) and dissociation rate constant k_{d} (or k_{off}), respectively. K_{D} is related to kₐ and k_{d} through the equation K_{D}=k_{d}/kₐ. Furthermore, “affinity” relates to the strength of the binding between a single binding site of a molecule (e.g., an antagonist) and a ligand (here receptor). The affinity of a molecule X for a ligand Y is represented by the dissociation constant (K_{d}), which is the concentration of Y that is required to occupy the combining sites of half the X molecules present in a solution. A smaller K_{d} indicates a stronger or higher affinity interaction, and a lower concentration of ligand is needed to occupy the sites. Similarly, the specificity of an interaction may be assessed by determination and comparison of the K_{D} value for the interaction of interest, such as a specific interaction between an antagonist and a receptor, with the K_{D} value of an interaction not of interest.

The term “significantly” is used to describe that an effect is of biological relevance, such as at least 10 or 20% inhibition or such as at least 10 or 20% induction.

The affinity of a C5aR antagonist may alternatively be determined in a competition ligand binding assay performed using neutrophils or mast cells. This functionality is referred to as affinity of the antagonist as measured in a competition assay, but could also be considered measurement of the avidity of the interaction. The ex-vivo assays measures the ability of C5aR antagonist to neutralize C5a mediated actions in an in-vitro setting. In one embodiment the C5aR antagonist has an affinity below 1.0 nM or 0.80 nM, such as below 0.50 nM or 0.35 nM, as measured by competition ligand binding assay on neutrophils.

A further functional characteristic of a C5aR antagonist is the ability to inhibit C5a-dependent migration of neutrophils. This functionality may be evaluated as described in Example 2 of WO2012/168199, the disclosure of which is incorporated herein by reference. Examples of antibodies displaying this functionality have been described in WO2012/168199. In one embodiment the invention thus relates to a C5aR antagonist, wherein said C5aR antagonist inhibits C5a induced cell migration (in vitro or in vivo).

In one embodiment a C5aR antagonist is capable of significantly inhibiting migration of human neutrophils.

In one embodiment the C5aR antagonist significantly inhibits migration of neutrophils in vitro.

In one embodiment the C5aR antagonist inhibits migration to less than 50%, less than 40%, less than 30%, less than 20%, or less than 10% compared to the level of migration observed in the presence of 10 nM C5a and no C5aR antagonist. In one such embodiment migration is measured after 30 minutes in the presence of 10 nM C5a and C5aR antagonist and compared to the level of migration observed after 30 minutes in the presence of 10 nM C5a and no C5aR antagonist. Alternatively the ability of a C5aR antagonist to inhibit neutrophil migration can be express using EC50 values based on the same set up. In one such embodiment the EC50 is below 2.5 μg/ml, such as below 2.5 μg/ml, such as below 1.5 μg/ml, such as below 1.2 μg/ml or even below 1.0 μg/ml.

A further method to determine in vitro the functionality of a C5aR antagonist is a calcium-flux assay, that measures the ability of an C5aR antagonist to inhibit C5a induced cellular (e.g. neutrophil) activation ex vivo, likewise described in Example 2 of WO2012/168199. In a further embodiment the invention relates to an C5aR antagonist with an EC50 as determined in a calcium-flux assay below 7.0 μg/ml, such as below 5.0 μg/ml, such as below 2.5 μg/ml.

Additional ex vivo assays can be used to determine the ability of a C5aR antagonist to inhibit or neutralize C5a induced neutrophil maturation based on secondary effects such as CD11 b and CD62L expression. CD11b and CD62L are maturation markers of neutrophils as they are up and down-regulated, respectively, upon activation by C5a/C5aR interaction.

In one embodiment, the invention relates to an C5aR antagonist with a EC50 as determined in an CD11 b up-regulation assay is below 3.0 μg/ml, such as below 2.0 μg/ml, such as below 1.5 μg/ml or such as 1.0 μg/ml or even below 0.5 μg/ml, between 0.3 μg/ml and 1.0 μg/ml, between 0.3 μg/ml and 2.0 μg/ml, between 0.3 μg/ml and 0.5 μg/ml, or about 0.5 μg/ml, or about 0.3 μg/ml.

Likewise, the effect of the C5aR antagonist in a CD62L down-regulation assay may be determined. In one embodiment, the invention relates to an C5aR antagonist with a EC50 as determined in a CD62L down-regulation assay is below 1.8 μg/ml, such as below 1.5 μg/ml, such as below 1.2 μg/ml or even below 1.0 μg/ml.

The skilled person will be aware of further criteria to determine if a given compound is a suitable C5aR antagonist and may thus choose an assay of preference within the scope of this invention. In vitro tests of C5aR antagonists are provided for example, in Example 2 of PCT publication no. WO2014/180961, the disclosure of which is incorporated herein by reference.

For example, an anti-C5aR antibody can be tested for its ability to inhibit up-regulation of CD11 b expression in response to C5a, according to the following materials and methods described herein in the Examples. In one assay, a C5aR antagonist can be tested for its ability to neutralize C5a-induced mast cell activation. In another assay, a C5aR antagonist can be tested for its ability to neutralize C5a-induced neutrophil activation. For example changes in CD11 b can be assessed using a CD11 b assay as further described herein, or CD62L expression can be measured using a CD11 b assay adapted for CD62L detection by using a conjugated antibody recognizing CD62L (BD Biosciences, Cat. No 559772), as described in PCT publication no. WO2014/180961.

In one embodiment the C5aR antagonist is an antibody binding the 2^{nd} loop of C5aR. In one embodiment the C5aR antagonist is an antibody binding the 2^{nd} loop (AA 175-206) of human C5aR (e.g., as shown in SEQ ID NO: 1). In on embodiment the C5aR antagonist is an antibody binding AA 179-186 (EEYFPPKV, SEQ ID NO: 12) of the human C5aR protein shown in as shown in SEQ ID NO: 1).

CDR sequences or variable regions of anti-C5aR antibodies are described in any one of WO 03/062278, WO/022390 and WO2012/168199. In any embodiment, a C5aR antagonistic antibody may be described as isolated to indicate that an antibody that has been separated and/or recovered from another/other component(s) of its natural environment and/or purified from a mixture of components in its natural environment.

Further examples of C5aR antagonist antibodies that binding the 2^{nd} loop of C5aR include antibodies having the heavy and light chain CDRs, variable regions and/or polypeptide chains of antibody 35F12A2, 35F24A3 and 35F32A3, as shown below.

35F12A2 variable regions and CDRs according to
Kabat numbering:
35F12A2 VH:
(SEQ ID NO: 13)
QVQLVESGGGVVQPGRSLRLSCVASGFTFSNYGMHWVRQAPGKGLEWVA
VIWYDGINKYYADSVKGRFTISRDNSKSTLYLQMNSLRAEDTAVYYCAG
TYYTSGSSKHFQPWGQGTLVTVSS.
VH CDR1:
(SEQ ID NO: 14)
NYGMH.
VH CDR2:
(SEQ ID NO: 15)
VIWYDGINKYYADSVK.
VH CDR3:
(SEQ ID NO: 16)
TYYTSGSSKHFQP.
35F12A2 VL:
(SEQ ID NO: 17)
EIVLTQSPATLSLSPGERATLSCRASQSVSSYLSWYQQKPGQAPRLLIY
NRATGIPARFSGSGSGTDFTLTISSLEPEDFAVYYCQQRSNWPTFGPGT
KVDIKR.
VL CDR1:
(SEQ ID NO: 18)
RASQSVSSYLS.
VL CDR2:
(SEQ ID NO: 19)
DASNRAT.
VL CDR3:
(SEQ ID NO: 20)
QQRSNWPT.
35F24A3 variable regions and CDRs according to
Kabat numbering:
(SEQ ID NO: 21)
35F24A3 VH:
EVKLVESGGGLVKPGGSLKLSCSASGFAFSNYDMSWVRQTPEKRLEWVA
AFSSDGYTFYPDSLKGRFTISRDNARNTLYLQMSSLGSEDTALYCCARH
ADYANYPVMDYWGQGTSVTVSS.
VH CDR1:
(SEQ ID NO: 22)
NYDMS.
VH CDR2:
(SEQ ID NO: 23)
AFSSDGYTFYPDSLK.
VH CDR3:
(SEQ ID NO: 24)
HADYANYPVMDY.
35F24A3 VL:
(SEQ ID NO: 25)
DIQMTQSPSSLSASVGDRVTITCRASQGISSWLAWYQQKPEKAPKSLIY
AASSLQSGVPSRFSGSGSGTDFTLTISSLQPEDFATYYCQQYNSYPRTF
GQGTKVEIKR.
VL CDR1:
(SEQ ID NO: 26)
RASQGISSWLA.
VL CDR2:
(SEQ ID NO: 27)
AASSLQS.
VL CDR3:
(SEQ ID NO: 28)
QQYNSYPRT.
35F32A3 variable regions and CDRs according to
Kabat numbering:
35F32A3 VH:
(SEQ ID NO: 29)
QVQLVESGGGLVRPGRSLRLSCAASGFTFRDYGMHWVRQAPGKSLEWVA
VIWFDGINKYYGDSVKGRFTISRDNSKNTLYLQMNSLRAEDTAMYYCVG
TYFGPGTTEFFQHWGQGTLVTVSS.
HCDR1:
(SEQ ID NO: 30)
DYGMH.
HCDR2:
(SEQ ID NO: 31)
VIWFDGINKYYGDSVK.
HCDR3:
(SEQ ID NO: 32)
TYFGPGTTEFFQH.
35F32A3 VL:
(SEQ ID NO: 33)
EIVLTQSPATLSLSPGERATLSCRASQSVSSYLAWYQQKPGQAPRLLPT
FGPGTKVDIKRIYDASNRATGIPARFSGSGSGTDFTLTISSLEPEDFAV
YYCQQRSNW.
CDRL1:
(SEQ ID NO: 34)
RASQSVSSYLA.
CDRL2:
(SEQ ID NO: 35)
DASNRAT.
CDRL3:
(SEQ ID NO: 36)
QQRSNWPT.

In one embodiment the C5aR antagonistic antibody is of the IgG isotype, such as IgG1, IgG2 or IgG4.

Antibodies, via the Fc domain, interact with various Fc receptors and it therefore relevant to consider if such interaction is favourable or not as described in WO2012/168199, such as an antibody with one or more Fc mutations selected from E233P, L234A or V234A or F234L or F234V, L235E or L235A, G236R or G236A, G237A, S239D, S254W, N297Q, L328R, A330S, P331S and 1332E. In one embodiment the antibody Fc is a human IgG1 domain including the mutations 234A, L235E, G237A, A330S and P331S (Kabat numbering). The antibody may additionally include a D327Q mutation.

In one embodiment the antibody according to the invention does not significantly induce phagocytosis of neutrophils in vitro, meaning that the level of phagocytosis is not significantly above background as measured in the absence of an anti-C5aR antibody. In one embodiment the antibody does not give rise to any detectable induction of phagocytosis. The assay for evaluating the level of phagocytosis may be performed using human neutrophils as described in Example 4 of WO2012/168199.

In an alternative assays the ability of anti-hC5aR antibodies to mediate cell depletion e.g. to induce ADCC (antibody dependent cellular cytotoxicity) and CDC (complement dependent cytotoxicity) may be evaluated. The assays apply C5aR expressing cells as target cells and effector cells (monocyte-depleted PMBCs) or complement containing sera to elicit the response. The assay is described further in Example 4 of WO2012/168199.

### Pharmaceutical Formulations

The present disclosure further includes pharmaceutical compositions and/or formulations, comprising a pharmaceutically acceptable carrier and a C5aR antagonist according to the disclosure, according to the dosages described herein.

The C5aR antagonist according to the disclosure may in an aspect of the invention be used in the preparation of a pharmaceutical composition. Such a pharmaceutical composition may be prepared based on general knowledge in the field such as in the Pharmacopeia or Remington.

In an embodiment the pharmaceutical composition according to the disclosure comprise an antibody as described herein in combination with a pharmaceutically acceptable carrier. The formulation may be in the form of a liquid formulation or a dry formulation that is reconstituted in water or an aqueous buffer composition prior to administration. The formulation may be in the form of an aqueous formulation. In an embodiment the formulation is sterilized.

A pharmaceutical composition of antibodies according to the disclosure may comprise a salt and/or buffer, such as the compositions described in WO2011/104381.

In further embodiment the pharmaceutical composition of an antibody according to the disclosure may be suitable for multiple uses, such as the compositions described in WO2011/147921.

In a further embodiment the pharmaceutical compositions of the C5aR antagonist may be for subcutaneous administration.

In a further embodiment the pharmaceutical compositions of the C5aR antagonist is for weekly administration.

### EMBODIMENTS

1. A method for treating an inflammatory skin disease, the method comprising administering an anti-C5aR antagonist antibody to the individual, by subcutaneous administration, optionally at a dose of 400-600 mg, optionally 450 mg, of at a frequency of once per week.
2. The method of embodiment 1, wherein the antibody is avdoralimab or a function-conservative variant thereof.
3. The method of any one of the above embodiments, wherein a plurality of doses of antibody are administered, and wherein each dose comprises the same amount of antibody.
4. The method of any one of the above embodiments, wherein the duration of the treatment is at least one month.
5. The method of any one of the above embodiments, wherein the duration of the treatment is between 1 month and 3 months, optionally between 1 month and 6 months.
6. The method of any one of the above embodiments, wherein the loading dose achieves a targeted blood concentration about 100 μg/ml on day of first administration.
7. The method of any one of the above embodiments, wherein a targeted blood concentration of about 100 μg/ml is maintained for the duration of the treatment.
8. The method of any one of the above embodiments, wherein the method is for treating or preventing an acute phase or an exacerbation of the inflammatory skin disease.
9. The method of any one of the above embodiments, wherein the inflammation is mediated by mast cells in the skin.
10. The method of any one of the above embodiments, wherein the inflammation is mediated by neutrophils in the skin.
11. The method of any one of the above embodiments, wherein the inflammatory skin disease is psoriasis, acne vulgaris, hidradenitis suppurativa (HS), systemic or cutaneous lupus erythematosus, cutaneous small vessel vasculitis, urticaria or urticarial vasculitis or bullous pemphigoid.
12. An anti-C5aR antagonist antibody, for use in treating an inflammatory skin disease, optionally wherein the inflammation is mediated by neutrophils and/or mast cells, wherein the antibody is administered by subcutaneous administration in an amount and schedule effective to: (i) achieve, within a 24, 12 or 6 hour period from first administration, a targeted blood concentration that provides at least EC₅₀ for inhibition of C5aR activity on neutrophils in tissues, optionally wherein the targeted blood concentration is 20 μg/ml, (ii) maintain a blood concentration that is at least the targeted blood concentration of step (i) for at least one week, and (iii) not exceed a targeted blood concentration that provides EC₁₀₀ for inhibition of C5aR activity on neutrophils in tissues (e.g. 100 μg/ml) by more than 2-fold or more than 50% during the treatment (e.g. for the duration of any 24 or 48 hour period).
13. The method of embodiment 12, wherein the antibody is avdoralimab and wherein the antibody is administered in a weekly dose of 450 mg.
14. A method inhibiting the activity of neutrophils and/or mast cells in an individual, the method comprising administering to the individual avdoralimab by subcutaneous administration at a dose of 400-600 mg, optionally 450 mg, optionally 6 mg/kg body weight, at a frequency of about once per week.
15. The method of any one of the above embodiments, wherein the dose achieves, within a 24, 12 or 6 hour period from first administration, a targeted blood concentration of antibody of about 20 μg/ml.
16. The method of embodiments 12-15, wherein starting from the fourth or fifth administration, a blood concentration of antibody of at least 20 μg/ml, optionally at least 50 μg/ml, optionally at least 70 or 80 μg/ml, is maintained for the duration of the treatment.
17. The method of embodiments 12-16, wherein the blood concentration of antibody not exceed the concentration that provides EC₁₀₀ for inhibition of C5aR activity on neutrophils in tissues (e.g. 100 μg/ml) by more than 2-fold or more than 50% during the treatment (e.g. for the duration of any 24 or 48 hour period).
18. The method of embodiments 12-17, wherein the method is for treating or preventing an acute phase or an exacerbation of the inflammatory skin disease.
19. The method of embodiments 12-18, wherein the inflammation is mediated by mast cells in the skin.
20. The method of embodiments 12-19, wherein the inflammation is mediated by neutrophils in the skin.
21. The method of embodiments 12-20, wherein the inflammatory skin disease is psoriasis, acne vulgaris, hidradenitis suppurativa (HS), systemic or cutaneous lupus erythematosus, cutaneous small vessel vasculitis, urticaria or urticarial vasculitis or bullous pemphigoid.
22. A method treating or preventing inflammation in an individual, the method comprising administering to the individual avdoralimab by subcutaneous administration as a loading dose of 450 mg once per week.
23. The method of embodiment 22, wherein the duration of the treatment is at least one month.
24. The method of embodiments 22-23, wherein the duration of the treatment is between 1 month and 3 months, optionally between 1 month and 6 months.
25. The method of embodiments 22-24, wherein at the fifth or sixth administration, the treatment provides and maintains a targeted blood concentration of at least 70 μg/ml, optionally a blood concentration of about 80, 90 or 100 μg/ml.
26. The method of embodiments 22-25, wherein following the fifth or sixth administration, a targeted blood concentration of at least 70 μg/ml, optionally about 80, 90 or 100 μg/ml, is maintained for the duration of the treatment.
27. The method of embodiments 22-26, wherein the method is for treating or preventing an acute phase or an exacerbation of the inflammatory skin disease.
28. The method of embodiments 22-27, wherein the inflammation is mediated by mast cells in the skin.
29. The method of embodiments 22-28, wherein the inflammation is mediated by neutrophils in the skin.
30. The method of embodiments 22-29, wherein the inflammatory skin disease is psoriasis, acne vulgaris, hidradenitis suppurativa (HS), systemic or cutaneous lupus erythematosus, cutaneous small vessel vasculitis, urticaria or urticarial vasculitis or bullous pemphigoid.
31. The method of any one of the preceding embodiments, wherein the treatment is for obtaining a fast response.
32. The method of any one of the preceding embodiments, wherein the method comprises a step of assessing an amelioration in inflammatory skin disease within 3 months, 2 months, 1 month or two weeks of first administration of the anti-C5aR antibody, and upon a determination of an amelioration in inflammatory skin disease, continuing treatment with the anti-C5aR antibody.
33. The method of any one of the preceding embodiments, wherein the antibody inhibits or reduces the C5aR mediated biological effect of C5a, such as
   a. C5a induced neutrophil activation,
   b. C5a induced mast cell activation,
   c. C5a induced cell migration and/or
   d. C5a induced neutrophil maturation.
34. The method of any one of the preceding embodiments, wherein the treatment inhibits or reduces the C5aR mediated biological effect of C5a in tissue, such as
   a. C5a induced neutrophil activation,
   b. C5a induced mast cell activation,
   c. C5a induced cell migration and/or
   d. C5a induced neutrophil maturation.
35. The method of any one of the preceding embodiments, wherein the antibody inhibits C5a-induced CD11 b expression on neutrophils, with an EC50 between 0.3 and 1 μg/ml, optionally with an EC50 below 1 μg/ml, optionally with an EC50 below 0.5 μg/ml, optionally with an EC50 between 0.3 and 0.5 μg/ml.
36. The method of any one of the preceding embodiments, wherein the antibody binds the 2^{nd} loop of C5aR.
37. The method of any of the previous embodiments, wherein the antibody inhibits C5a signaling via C5aR.
38. The method of any of the previous embodiments, wherein the antibody inhibits or reduces binding of C5a to C5aR.
39. The method of any of the previous embodiments, wherein the antibody displaces C5a in an SPA assay, with an EC₅₀ below 50 nM.
40. The antibody for use according to any of the previous embodiments, wherein the affinity of the antibody as measured in a competition ligand binding assay on neutrophils is below 0.80 nM.
41. The method of any of the previous embodiments, wherein the antibody neutralizes C5a induced neutrophil activation ex vivo with an EC₅₀ as determined in a calcium-flux assay below 7.0 μg/ml.
42. The method of any of the previous embodiments, wherein the antibody inhibits C5a induced cell migration.
43. The method of any of the previous embodiments, wherein the antibody significantly inhibits migration of neutrophils in vitro.
44. The method of any of the previous embodiments, wherein the antibody reduces neutrophil migration to less than 50%.
45. The method of any of the above embodiments, wherein the antibody reduces neutrophil migration with and EC₅₀ below 2.5 μg/ml.
46. The method of any of the previous embodiments, wherein the antibody inhibits C5a induced neutrophil maturation ex vivo.
47. The method of any of the previous embodiments, wherein the antibody inhibits C5a induced neutrophil maturation ex vivo with;
   a. an EC₅₀ as determined in a CD11b up-regulation assay below 3.5 μg/ml, optionally below 2.5 μg/ml, optionally below 1.5 μg/ml optionally below 1.0 μg/m or
   b. an EC₅₀ as determined in a CD62L down-regulation assay below 1.8 μg/ml, optionally below 1.5 μg/ml, optionally below 1.2 μg/ml or optionally below 1.0 μg/ml.
48. The method of any of the previous embodiments wherein the C5aR antagonist is an antibody which do not significantly induce phagocytosis of neutrophils in vitro.
49. The method of any of the previous embodiments wherein the antibody which does not significantly induce ADCC in vitro.
50. The method of any of the previous embodiments wherein antibody does not significantly induce CDC in vitro.
51. The method of any of the preceding embodiments, wherein the antibody has the heavy chain CDRs of SEQ ID NOS: 5, 6 and 7 and the light chain CDRs of SEQ ID NOS: 9, 10 and 11.
52. The method of any of the preceding embodiments, wherein the antibody is avdoralimab or a function-conservative variant thereof.
53. A pharmaceutical composition for weekly subcutaneous administration, wherein the composition comprises avdoralimab in an amount of 450 mg.
54. A kit comprising a plurality of doses of antibody according to any of the above embodiments.

The invention may further be described by, but not limited to, the Embodiments described here below. The findings are also illustrated by the Examples presented herein.

### EXAMPLES

### Example 1: Receptor Saturation of Avdoralimab in a Human Phase I Clinical Trial

Avdoralimab was explored in a dose-escalation phase I trials. A total of 45 patients received avdoralimab and 15 patients received placebo with no safety concerns raised in any of the two trials.

In the single dose administration trial, 36 patients with rheumatoid arthritis (RA) received a single dose of either placebo (n=9) or avdoralimab, at 0.02, 0.08, 0.3, 1, 3 or 10 mg/kg (i.v., n=18) or avdoralimab at 0.2, 0.8 or 4 mg/kg by subcutaneous administration (s.c, n=9). The trial duration was 10 weeks and consisted of a 4-week screening period, a dosing visit at week 1 and a 9-week follow-up period.

In the multiple dose administration trial, twenty-four (24) patients with rheumatoid arthritis received multiple doses of either placebo (n=6) or avdoralimab, at 0.25 mg/kg (s.c., n=6) or at 1.0 mg/kg (s.c., n=6) or at 4.0 mg/kg (s.c., n=6). Subjects were dosed weekly for a total of 7 weeks and then attended 6 follow-up visits until 13 weeks after last treatment dose. The total study duration was approximately 19 weeks for each subject.

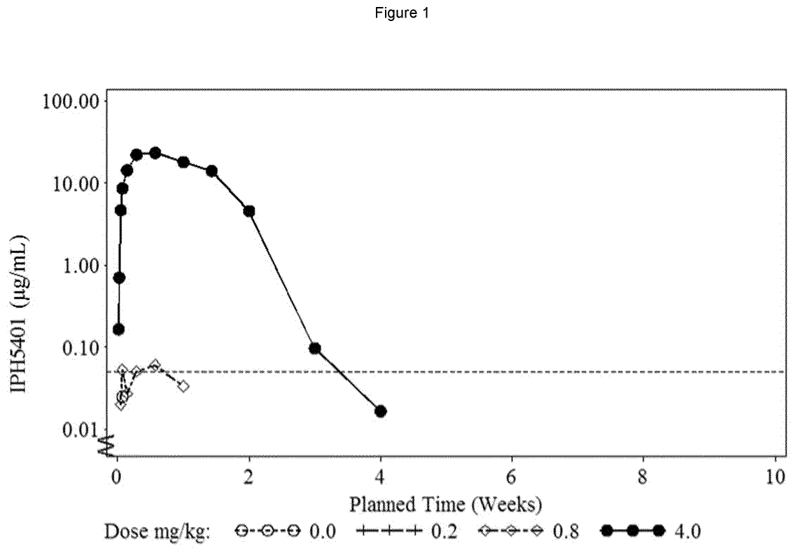

FIG. 1 shows the mean avdoralimab (IPH5401) serum concentration (μg/mL) over time after single s.c. dosing of subjects with RA. LLoQ=0.05 μg/mL. All samples below LLOQ were set to 0. Concentrations were above LLOQ for subjects at the highest dose level of 4.0 mg/kg, and for a single subject at 0.8 mg/kg. The mean PK parameters could be calculated only for the 4 mg/kg cohort. The absorption was relatively slow, with an observed mean Tmax of 4.0 days (SD=0.1). The mean AUC for s.c. cohort 4 mg/kg was 4930 μg·h/mL (51.9% CV). The terminal half-life could not be interpreted due to the nonlinear PK.

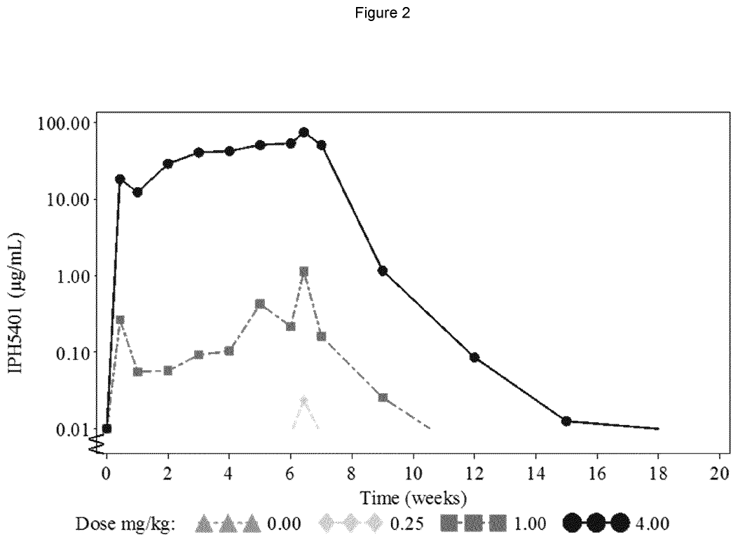

FIG. 2 shows the mean avdoralimab serum concentration (μg/mL) over time after multiple s.c. dosing of subjects with RA. Systemic exposure to avdoralimab increased throughout the dosing period, with increasing dose levels. The difference in the steady-state serum levels of avdoralimab between the 1 and 4 mg/kg cohorts was much larger than the 4-fold difference in dose, indicating nonlinear PK. Steady state was reached after 5 weeks administrations and Ctau was on average 50 μg/mL at week 5 to 7 at the 4 mg/kg dose level.

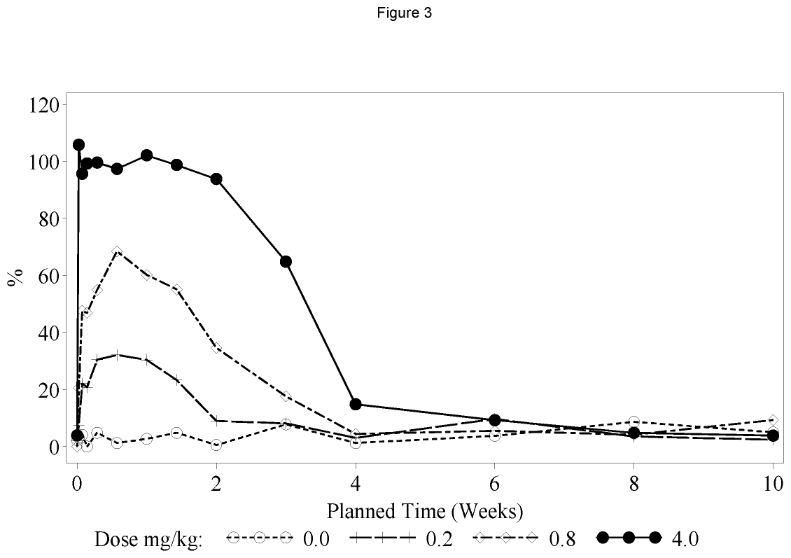

FIG. 3 shows the mean C5aR occupancy (%) on neutrophils over time by s.c. dose levels (single s.c. administration). After a single s.c. dose of 4.0 mg/kg, full receptor occupancy was maintained for an average of 1.8 weeks and declined to below 20% at an average of 5.5 weeks after a single injection.

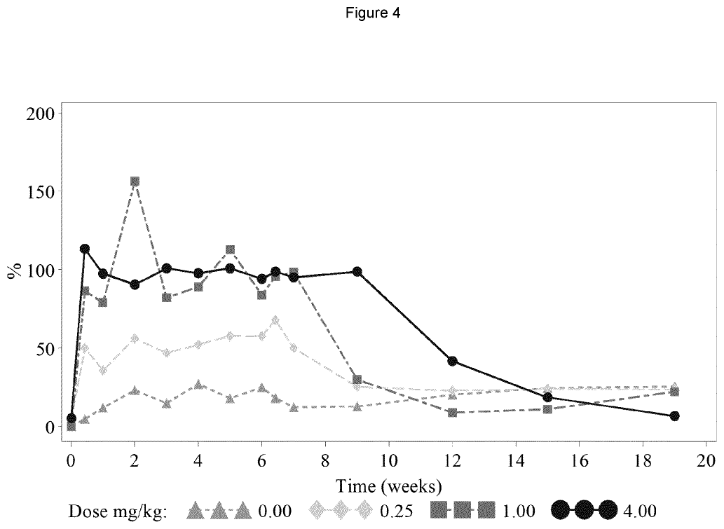

FIG. 4 shows the mean C5aR occupancy (%) on neutrophils and granulocytes over time by s.c. dose levels (multiple s.c. administrations). After multiple s.c. doses, full receptor occupancy was achieved in subjects treated with 1 and 4 mg/kg and was maintained for an average of 9 weeks in the highest dose cohort.

Overall, full receptor occupancy was achieved in the s.c. single dose study for subjects treated at the highest dose 4 mg/kg for an average of 1.8 week duration and was achieved for both 1 mg/kg and 4 mg/kg dose groups in the multiple s.c. dose study for an average of 9 week duration in the latter group.

### Example 2: Development of a PKPD Model for Avdoralimab

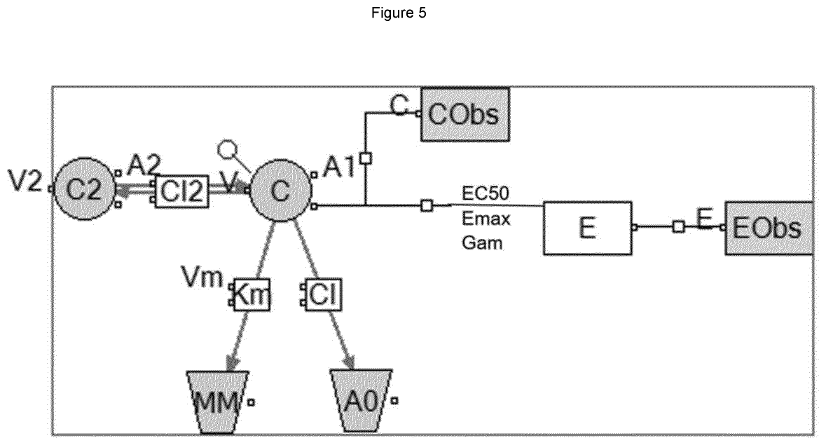

A PKPD model was built in Phoenix 64 WinNonLin version 8.1.0.3530, to predict avdoralimab serum concentrations in blood post s.c. administration, as well as C5aR saturation. Pharmacokinetics of therapeutic mAbs are usually modelled using a two-compartment model (Deng et al. 2011 MAbs 3(1): 61-66). Based on the PK results described in Example 1, avdoralimab is expected to display PK properties similar to other therapeutic antibodies in humans, except for compound-specific target-mediated drug disposition (TMDD). This TMDD effect can be modelled by an additional non-linear elimination added to the model (Wang et al. 2016 Biopharm. Drug Dispos. 37: 51-65). A two-compartment model with parallel first order (linear) and saturable (non-linear, Michaelis-Menten) elimination from the central compartment was developed to adequately describe the observed PK of avdoralimab following repeated s.c. administration in humans, as illustrated in FIG. 5, and using the parameter values listed in Table 1 below. Absorption rate (Ka) and bioavailability (Ba) for s.c. administration were estimated based on PK results for Example 1. EC₅₀ in the PD part of PKPD model is derived from PK/RSA data observed in phase I clinical trial, and is similar to EC₅₀ binding to human neutrophils in vitro=0.34 nM=51 ng/mL in FACS titration assays.

TABLE 1

| Parameter | Value | Unit |
|---|---|---|
| CL (dose dependent) | 0.3 | mL/h |
| V | 40 | mL/kg |
| V2 | 30 | mL/kg |
| Cl2 | 0.2 | mL/h |
| Vm | 5000 | ng/h |
| Km | 30 | ng/ml |
| EC50 | 50 | ng/ml |
| Emax | 100 | % |
| Ka | 0.00763 | 1/h |
| Ba | 0.775 | — |

### Example 3: Design of a Multiple Dose Avdoralimab Treatment Regimen for Subcutaneous Administration

### Capacity of Avdoralimab to Inhibit Up-Regulation of CD11 b Expression in Human Neutrophils

Avdoralimab was assessed for its ability to neutralize C5a-induced neutrophil maturation by measuring changes in CD11 b expression.

Peripheral venous blood was collected into heparin-coated tubes from healthy volunteers. After collection 35 μL of blood were added to 35 μL of avdoralimab (IPH5401) at different concentrations in PBS in a culture-treated 96-well plate U-bottom (BD Falcon, 353077), homogenized by pipetting, and incubated for 20 min. at +37±3° C.+5±1% CO2. After incubation, 50 μL of the blood incubated with Avdoralimab are transferred in a new 96-well plate containing 10 μL of 18 nM human recombinant C5a (R&D systems) in PBS per well. Each well was homogenized by pipetting, and the plates were incubated for 20 min. at +37±3° C.+5±1% CO2.

Then 50 μL of each sample was stained for flow cytometry using 25 μl of a mastermix containing saturating concentrations of anti-CD16 FITC (BD Biosciences, 556616) and anti-CD11 b PE-Cy5 (BD Biosciences) in a staining buffer containing PBS (Lonza), 2 mM EDTA (Invitrogen), 0.2% BSA (Sigma, A9418), and 0.02% sodium azide (Sigma, 71290-100 g). Following incubation in the dark for 20 min. at +37±3° C.+5±1% CO2 erythrocytes are lysed by adding 500 μL of Optilyse C solution (Beckman Coulter, A11895) and incubating for 10 min. in the dark at RT, then by adding 500 μL of PBS (Lonza) and incubating for 10 min. in the dark at RT. Cells are washed using PBS (Lonza), and resuspended in CytoFix (BD Bioscience) for fixation at 4° C. in the dark during 30 min. and subsequently analyzed on a LSRII flow cytometer (BD Biosciences) using FACS Diva software. Cells are finally washed in staining buffer, resuspended in staining buffer, and analyzed on a FACS Canto II flow cytometer (BD Biosciences) using FACS Diva software.

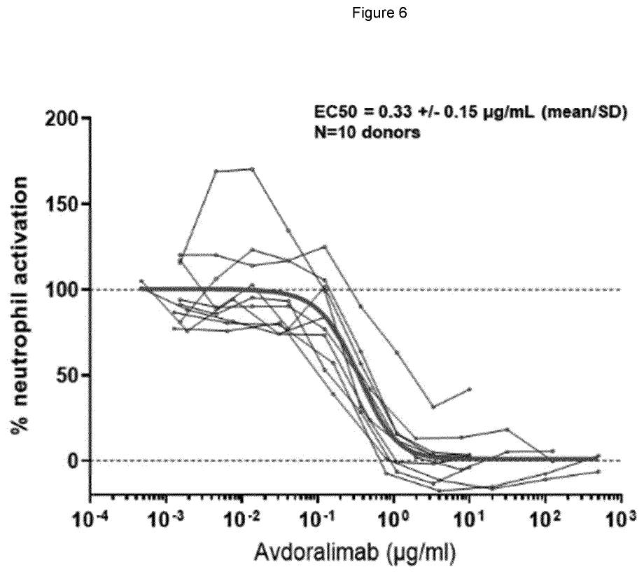

Results: C5a-induced CD11 b expression on neutrophils (defined as SSChigh CD16+ cells) was quantified as median fluorescence intensity (MedFI) of PE-Cy5 for a given Avdoralimab concentration. The MedFI were normalized, with 0% being the value obtained from blood where no C5a was added, and 100% the value obtained from blood with C5a and no avdoralimab. Results are shown in FIG. 6. The EC₅₀ value for Avdoralimab was calculated as 0.34 μg/mL using 5PL non-linear curve fitting in GraphPad Prism software (v. 7). The EC₁₀₀ value for Avdoralimab was around 2 μg/mL.

The concentrations of avdoralimab required for binding and saturation of C5aR on cells from Example 1 were compared to the concentrations required for inhibition of C5aR as assessed by measuring C5a-induced CD11 b expression on neutrophils. In assays for inhibition of C5aR, avdoralimab displayed an in vitro EC₅₀ of 0.34 μg/mL and an EC₁₀₀ of about 2 μg/mL. Additionally, the overall in vitro EC₅₀ values ranged from about 0.5 to about 2 μg/mL in a panel of four assays considered (Calcium Flux, CD11b upregulation, CD62L down-regulation, and neutrophils migration).

The in vitro EC₅₀ in the C5aR inhibition assays was therefore found to be about 6× to 20× higher than the EC₅₀ of saturation. It therefore appears in order to fully inhibit C5aR in competition with C5a, avdoralimab should be provided in an amount that provides for a several-fold greater concentration than that which provides 100% saturation of C5aR.

Based on these findings, the targeted blood concentration for inhibition of neutrophils would be around 20 μg/ml, corresponding to about the EC₁₀₀ of in vitro efficacy assays, and further that the targeted blood concentration sufficient to achieve optimal concentration of avdoralimab for inhibition of neutrophils in tissues (e.g., skin) is about 100 μg/ml.

### A Regimen for Long Term Avdoralimab Treatment, Multiple Dose (MD), Subcutaneous Administration (sc)

PD/PK simulations were performed using the software Phoenix WinNonLin version 8.1.0.3530 and plotting of the results was done in GraphPad Prism 8 version 8.0.2 (263). The model described in Example 2 was implemented in WinNonLin and used to simulate the PK over time following subcutaneous (s.c.) administration of avdoralimab to humans for a range of dose levels, with weekly administrations. Based on this, doses and schedule were identified that would permit maintenance of the target C_{trough} at steady state.

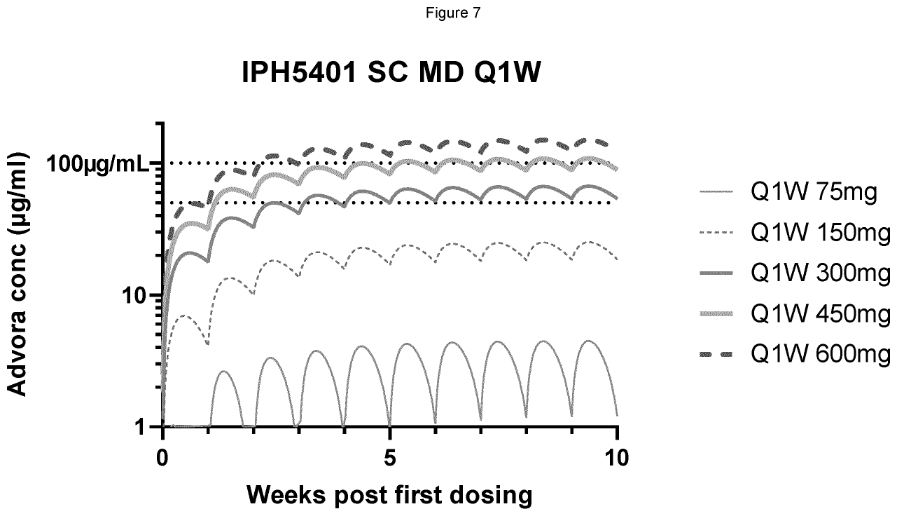

PD/PK simulations were performed to identify doses for weekly s.c. administration of avdoralimab that achieve and maintain the targeted blood concentration of about 100 μg/ml. Results showed that the targeted blood concentration can be achieved from a dose of 6 mg/kg body weight based dose (equal to a 450 mg flat dose for a 75 kg individual), administered every week (Q1w). FIG. 7 shows results of PK prediction for Q1w for the 75 mg, 150 mg, 300 mg, 450 mg, 600 mg flat doses (for a 75 kg individual).

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). Where “about” is used in connection with a number, this can be specified as including values corresponding to +/−10% of the specified number.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1-30. (canceled)

31. A method for treating an inflammatory disease, the method comprising administering avdoralimab to the individual, by subcutaneous administration, at a dose of 400-600 mg, at a frequency of once per week.

32. The method of claim 31, wherein a plurality of doses of avdoralimab are administered, and wherein each dose comprises 450 mg of antibody.

33. The method of claim 31, wherein the duration of the treatment is at least one month.

34. The method of claim 31, wherein the duration of the treatment is between 1 month and 6 months.

35. The method of claim 31, wherein a targeted blood concentration of avdoralimab of about 100 μg/ml is maintained for the duration of the treatment.

36. The method of claim 31, wherein the method is for treating or preventing an acute phase or an exacerbation of an inflammatory skin disease.

37. The method of claim 31, wherein the inflammation is mediated by mast cells in the skin.

38. The method of claim 31, wherein the inflammation is mediated by neutrophils in the skin.

39. The method of claim 31, wherein the inflammatory disease is an inflammatory skin disease.

40. The method of claim 39, wherein the inflammatory skin disease is psoriasis, acne vulgaris, hidradenitis suppurativa (HS), systemic or cutaneous lupus erythematosus, cutaneous small vessel vasculitis, urticaria or urticarial vasculitis or bullous pemphigoid.

41. The method of claim 31, wherein the method comprises a step of assessing an amelioration in inflammatory skin disease within 3 months of first administration of avdoralimab, and upon a determination of an amelioration in inflammatory skin disease, continuing treatment with avdoralimab.

42. A method inhibiting the activity of neutrophils and/or mast cells in an individual, the method comprising administering to the individual avdoralimab by subcutaneous administration at a dose of 400-600 mg, at a frequency of about once per week.

43. The method of claim 42, wherein avdoralimab is administered at a weekly dose of 450 mg.

44. The method of claim 42, wherein the individual has an inflammatory skin disease selected from psoriasis, acne vulgaris, hidradenitis suppurativa (HS), systemic or cutaneous lupus erythematosus, cutaneous small vessel vasculitis, urticaria or urticarial vasculitis or bullous pemphigoid.

45. A pharmaceutical composition for weekly subcutaneous administration, wherein the composition comprises avdoralimab in an amount of 450 mg.

46. A kit comprising a plurality of doses of avdoralimab according to claim 45.


SEQUENCE LISTING (36 sequences)

SEQ ID NO: 1 - protein, 350 aa, homo sapiens
MDSFNYTTPDYGHYDDKDTLDLNTPVDKTSNTLRVPDILALVIFAVVFLVGVLGNALVVWVTAFEAKRTINAIWFLNLAVADFLSCLALPILFTSIVQHHHWPFGGAACSILPSLILLNMYASILLLATISADRFLLVFKPIWCQNFRGAGLAWIACAVAWGLALLLTIPSFLYRVVREEYFPPKVLCGVDYSHDKRRERAVAIVRLVLGFLWPLLTLTICYTFILLRTWSRRATRSTKTLKVVVAVVASFFIFWLPYQVTGIMMSFLEPSSPTFLLLKKLDSLCVSFAYINCCINPIIYVVAGQGFQGRLRKSLPSLLRNVLTEESVVRESKSFTRSTVDTMAQKTQAV

SEQ ID NO: 2 - protein, 454 aa, artificial
  synthetic
EVQLVESGGGLVQPGGSLRLSCAASGFTFSSYVMHWVRQATGKGLEWVSAIDTGGGTYYADSVKGRFTISRENAKNSLYLQMNSLRAGDTAVYYCARDYYYYASGSYYKAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, artificial
  synthetic
EIVLTQSPGTLSLSPGERATLSCRASQSVSSRYLAWYQQKPGQAPRLLIYGASSRATGIPDRFSGSGSGTDFTLTISRLEPEDFAVYYCQQYGSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 124 aa, artificial
  synthetic
EVQLVQSGGGLVHPGGSLRLSCAGSGFTFSSYVMHWVRQAPGKGLEWVSAIDTGGGTYYADSVKGRFTISRDNAKNSLYLQMNSLRAEDMAVYYCARDYYYYASGSYYKAFDIWGQGTMVTVSS

SEQ ID NO: 5 - protein, 5 aa, Artificial
  Synthetic
SYVMH

SEQ ID NO: 6 - protein, 15 aa, Artificial
  Synthetic
AIDTGGGTYYADSVK

SEQ ID NO: 7 - protein, 16 aa, Artificial
  Synthetic
DYYYYASGSYYKAFDI

SEQ ID NO: 8 - protein, 107 aa, Artificial
  Synthetic
EIVLTQSPGTLSLSPGERATLSCRASQSVSSRYLAWYQQKPGQAPRLLIYGASSRATGIPDRFSGSGSGTDFTLTISRLEPEDFAVYYCQQYGSPLTFGQGTKLEIK

SEQ ID NO: 9 - protein, 12 aa, Artificial
  Synthetic
RASQSVSSRYLA

SEQ ID NO: 10 - protein, 7 aa, Artificial
  Synthetic
GASSRAT

SEQ ID NO: 11 - protein, 8 aa, Artificial
  Synthetic
QQYGSPLT

SEQ ID NO: 12 - protein, 8 aa, homo sapiens
EEYFPPKV

SEQ ID NO: 13 - protein, 122 aa, Artificial
  Synthetic
QVQLVESGGGVVQPGRSLRLSCVASGFTFSNYGMHWVRQAPGKGLEWVAVIWYDGINKYYADSVKGRFTISRDNSKSTLYLQMNSLRAEDTAVYYCAGTYYTSGSSKHFQPWGQGTLVTVSS

SEQ ID NO: 14 - protein, 5 aa, Artificial
  Synthetic
NYGMH

SEQ ID NO: 15 - protein, 16 aa, Artificial
  Synthetic
VIWYDGINKYYADSVK

SEQ ID NO: 16 - protein, 13 aa, Artificial
  Synthetic
TYYTSGSSKHFQP

SEQ ID NO: 17 - protein, 107 aa, Artificial
  Synthetic
EIVLTQSPATLSLSPGERATLSCRASQSVSSYLSWYQQKPGQAPRLLIYDASNRATGIPARFSGSGSGTDFTLTISSLEPEDFAVYYCQQRSNWPTFGPGTKVDIKR

SEQ ID NO: 18 - protein, 11 aa, Artificial
  Synthetic
RASQSVSSYLS

SEQ ID NO: 19 - protein, 7 aa, Artificial
  Synthetic
DASNRAT

SEQ ID NO: 20 - protein, 8 aa, Artificial
  Synthetic
QQRSNWPT

SEQ ID NO: 21 - protein, 120 aa, Artificial
  Synthetic
EVKLVESGGGLVKPGGSLKLSCSASGFAFSNYDMSWVRQTPEKRLEWVAAFSSDGYTFYPDSLKGRFTISRDNARNTLYLQMSSLGSEDTALYCCARHADYANYPVMDYWGQGTSVTVSS

SEQ ID NO: 22 - protein, 5 aa, Artificial
  Synthetic
NYDMS

SEQ ID NO: 23 - protein, 15 aa, Artificial
  Synthetic
AFSSDGYTFYPDSLK

SEQ ID NO: 24 - protein, 12 aa, Artificial
  Synthetic
HADYANYPVMDY

SEQ ID NO: 25 - protein, 108 aa, Artificial
  Synthetic
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIKR

SEQ ID NO: 26 - protein, 11 aa, Artificial
  Synthetic
RASQGISSWLA

SEQ ID NO: 27 - protein, 7 aa, Artificial
  Synthetic
AASSLQS

SEQ ID NO: 28 - protein, 9 aa, Artificial
  Synthetic
QQYNSYPRT

SEQ ID NO: 29 - protein, 122 aa, Artificial
  Synthetic
QVQLVESGGGLVRPGRSLRLSCAASGFTFRDYGMHWVRQAPGKSLEWVAVIWFDGINKYYGDSVKGRFTISRDNSKNTLYLQMNSLRAEDTAMYYCVGTYFGPGTTEFFQHWGQGTLVTVSS

SEQ ID NO: 30 - protein, 5 aa, Artificial
  Synthetic
DYGMH

SEQ ID NO: 31 - protein, 16 aa, Artificial
  Synthetic
VIWFDGINKYYGDSVK

SEQ ID NO: 32 - protein, 13 aa, Artificial
  Synthetic
TYFGPGTTEFFQH

SEQ ID NO: 33 - protein, 107 aa, Artificial
  synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGPGTKVDIKR

SEQ ID NO: 34 - protein, 11 aa, Artificial
  synthetic
RASQSVSSYLA

SEQ ID NO: 35 - protein, 7 aa, Artificial
  synthetic
DASNRAT

SEQ ID NO: 36 - protein, 8 aa, Artificial
  synthetic
QQRSNWPT